(19) United States
(12) Patent Application Publication
(10) Pub. No.: US 2025/0312050 A1
(43) Pub. Date: Oct. 9, 2025

(54) GUIDE DEVICE

(71) Applicant: OLYMPUS TERUMO BIOMATERIALS CORP., Tokyo (JP)

(72) Inventors: Nobuyoshi WATANABE, Kyoto (JP); Shu IHARA, Tokyo (JP)

(73) Assignee: OLYMPUS TERUMO BIOMATERIALS CORP., Tokyo (JP)

(21) Appl. No.: 19/240,261

(22) Filed: Jun. 17, 2025

Related U.S. Application Data
(63) Continuation of application No. PCT/JP2022/046641, filed on Dec. 19, 2022.

(51) Int. Cl. A61B 17/17; A61B 17/02

(52) U.S. Cl. CPC A61B 17/1764; A61B 2017/0268

(57) ABSTRACT

A guide device includes: a guide portion guiding a bone cutting tool; a retractor portion arranged so as to be spaced apart from the guide portion in a first direction; a connection portion that connects the guide portion and the retractor portion; and a fixing portion that fixes the guide portion with respect to the tibia. The guide portion has a guide hole penetrating the guide portion in the first direction and extending in a second direction intersecting the first direction. The retractor portion is disposed in a region overlapping at least the guide hole in a plan view in the first direction.

### CROSS-REFERENCE TO RELATED APPLICATIONS

This is a continuation of International Application PCT/JP2022/046641, with an international filing date of Dec. 19, 2022, which is hereby incorporated by reference herein in its entirety.

### TECHNICAL FIELD

The present disclosure relates to a guide device, and in particular, relates to a guide device for a vertical osteotomy in TCVO.

### BACKGROUND

In advanced medial knee osteoarthritis, joint instability known as the teeter effect may occur due to wear of cartilage and bones in the medial femorotibial joint. For the purpose of improving this symptom, tibial condylar valgus osteotomy (TCVO) is performed.

As shown in FIG. 14, TCVO is a surgical procedure for changing the shape of an articular surface B by performing an L-shaped osteotomy in a proximal end portion of the tibia A, and can improve the congruency of the articular surface B, thereby shifting the load line (see the broken line) outward. In TCVO, a lateral osteotomy is performed from the medial cortical bone of the tibia A to the medial border of the tibial tuberosity, and a vertical osteotomy is performed from the medial border of the tibial tuberosity to the lateral intercondylar eminence or the center of the intercondylar eminence. In FIG. 14, reference sign C indicates the femur and reference sign D indicates the fibula.

In an osteotomy such as TCVO, there are cases in which a guide device for guiding a bone cutting instrument, such as a bone saw, is used in order to accurately and easily cut the bone (for example, see Patent Literature 1).

### SUMMARY

An aspect of the present disclosure is a guide device comprising: a guide portion that is disposed on a front side of the tibia, the guide portion guiding a bone cutting tool for cutting the tibia; a retractor portion disposed on a back side of the tibia, the retractor portion being arranged so as to be spaced apart from the guide portion in a first direction; a connection portion that connects the guide portion and the retractor portion; and a fixing portion that fixes the guide portion with respect to the tibia, wherein the guide portion has a guide hole into which the bone cutting tool is inserted, the guide hole penetrating the guide portion in the first direction and extending in a second direction intersecting the first direction, and the retractor portion is disposed in a region overlapping at least the guide hole in a plan view in the first direction.

### BRIEF DESCRIPTION OF DRAWINGS

FIG. 1 is an assembly perspective view of a guide device according to one embodiment.

FIG. 2A is a perspective view of a first guide portion.

FIG. 2B is a perspective view of a second guide portion.

FIG. 3A is a cross-sectional view of a retractor portion.

FIG. 3B is a longitudinal sectional view of a retractor portion.

FIG. 4 is an example of an X-ray fluoroscopic image of the guide portion and the retractor portion that are disposed on the tibia.

FIG. 5 is a configuration diagram of an example of a lock mechanism.

FIG. 6 is a configuration diagram of another example of the lock mechanism.

FIG. 7 is a diagram for explaining a method of using the guide device in TCVO.

FIG. 8 is a diagram for explaining the method of using the guide device in TCVO.

FIG. 9 is a diagram for explaining the method of using the guide device in TCVO.

FIG. 10 is a diagram for explaining the method of using the guide device in TCVO.

FIG. 11 is a diagram for explaining the method of using the guide device in TCVO.

FIG. 12A is an assembly perspective view of a first unit and a second unit for a left leg.

FIG. 12B is an assembly perspective view of the first unit and a second unit for a right leg.

FIG. 13A is a diagram showing a holding portion when used for a left leg.

FIG. 13B is a diagram showing a holding portion when used for a right leg.

FIG. 14 is a diagram for explaining TCVO.

### DESCRIPTION OF EMBODIMENT

A guide device according to an embodiment of the present disclosure will be described below with reference to the drawings.

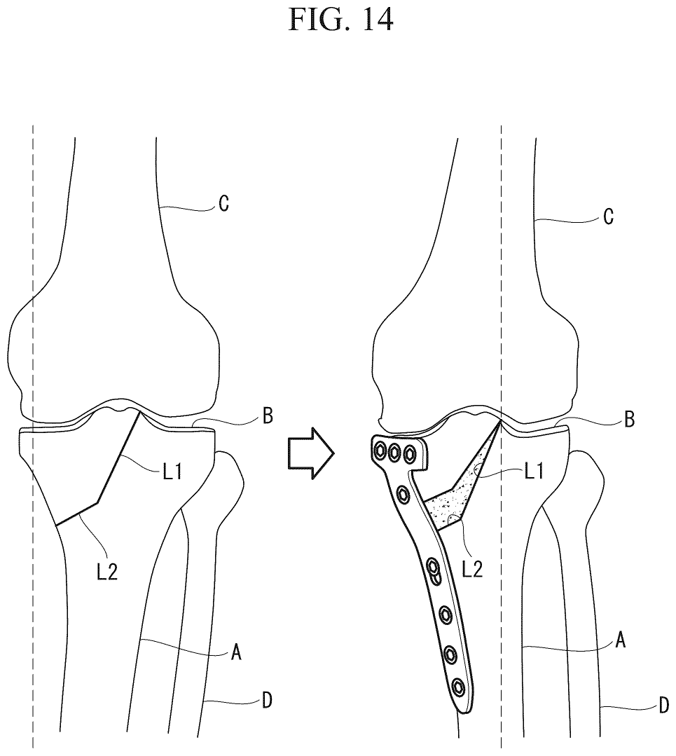

A guide device 1 according to this embodiment is for guiding a bone cutting tool and guide pins that are used in TCVO. As shown in FIG. 14, in TCVO, a proximal end portion of the tibia A is subjected to an L-shaped osteotomy by means of a vertical osteotomy and a lateral osteotomy. In FIG. 14, reference sign L1 indicates a vertical osteotomy line and reference sign L2 indicates a lateral osteotomy line. The guide device 1 is particularly used in the vertical osteotomy.

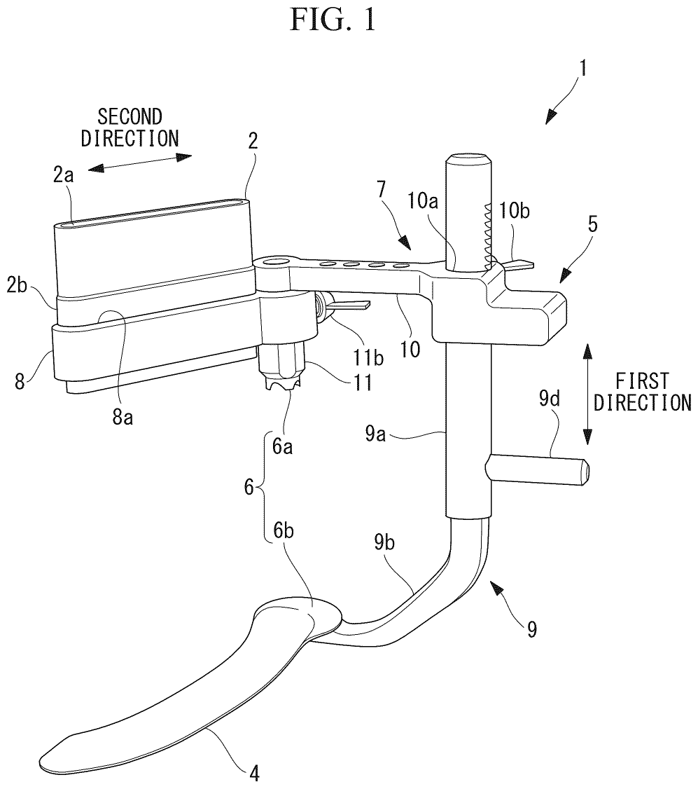

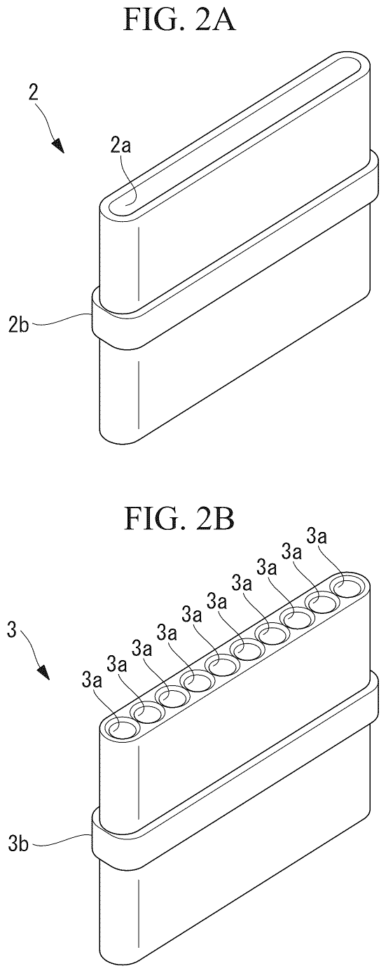

As shown in FIGS. 1, 2A, and 2B, the guide device 1 includes: two types of guide portions 2, 3 that are alternatively used; a retractor portion 4 that is arranged so as to be spaced apart from the guide portion 2 or 3 in a first direction; a connection portion 5 that connects the guide portion 2 or 3 and the retractor portion 4; a fixing portion 6 that fixes the guide portion 2 or 3 with respect to the tibia A; and an adjustment portion 7 that adjusts the position of the fixing portion 6.

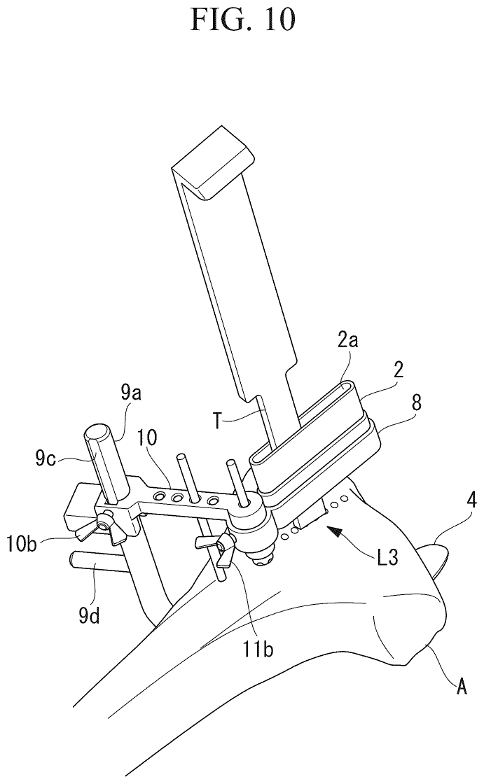

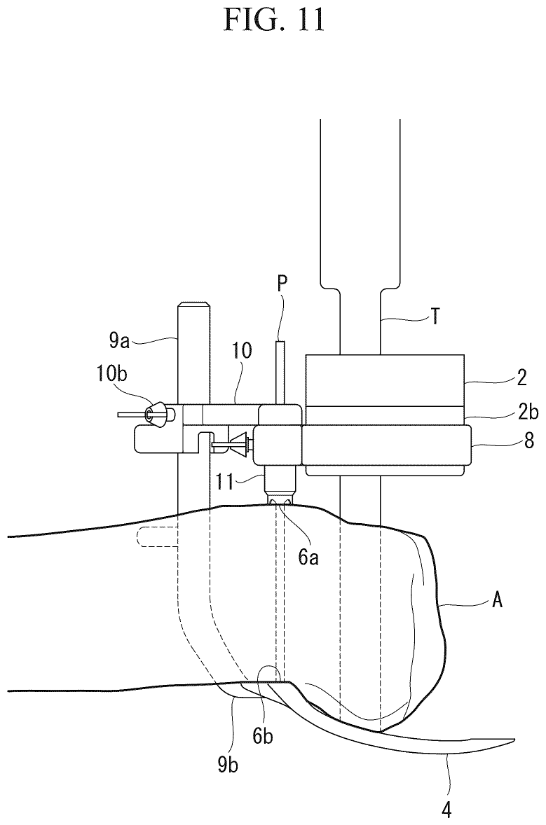

When TCVO is performed, the front-back direction and the longitudinal direction of the tibia A are respectively arranged in a substantially vertical direction and a substantially horizontal direction, and a front surface and a back surface of the tibia A are respectively arranged on the upper side and the lower side. Soft tissue, such as the popliteal artery and vein and the tibial nerve, is present on the back side of the tibia A. The vertical osteotomy in the tibia A is performed by means of a bone cutting tool T from the front surface toward the back surface (see FIGS. 10 and 11).

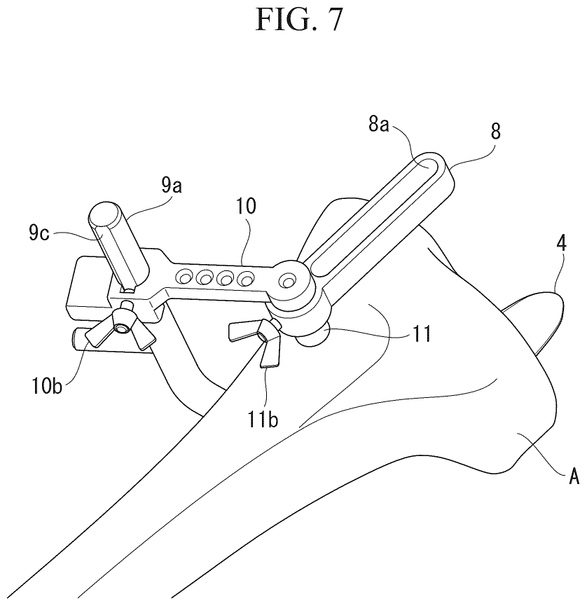

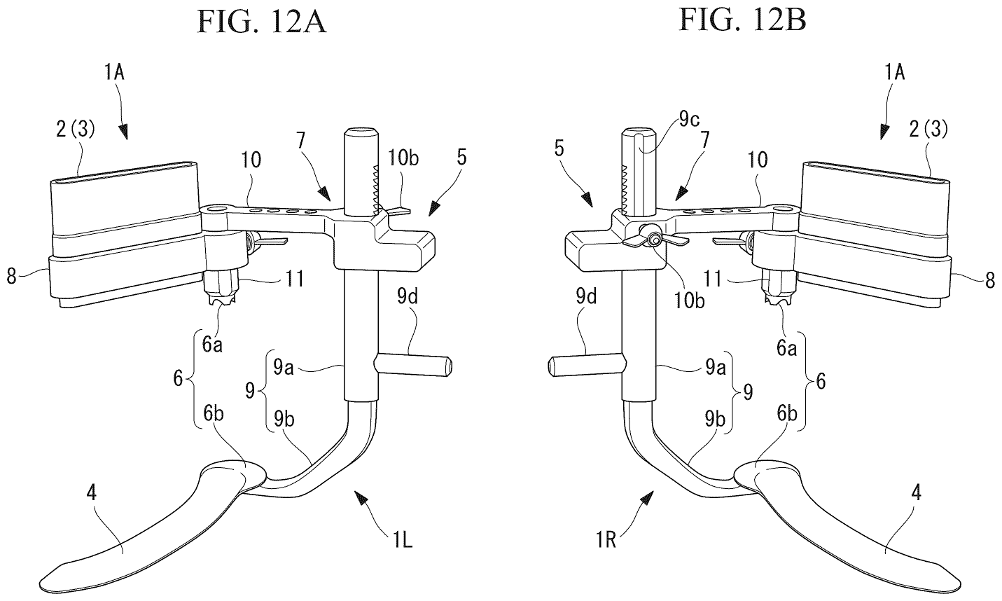

As shown in FIGS. 7 to 11, the guide portion 2, 3 is disposed on the front side of the tibia A, the retractor portion 4 is disposed on the back side of the tibia A, and the connection portion 5 is disposed on the inner side of the tibia A. Therefore, the first direction corresponds to the front-back direction and the vertical direction of the tibia A, and a second direction, which will be described later, corresponds to the longitudinal direction and the horizontal direction of the tibia A. The guide device 1 in FIG. 1 is for a left leg. A guide device for a right leg has a structure symmetrical to the guide device 1 in FIG. 1 with respect to a plane in the first direction (see FIG. 12B).

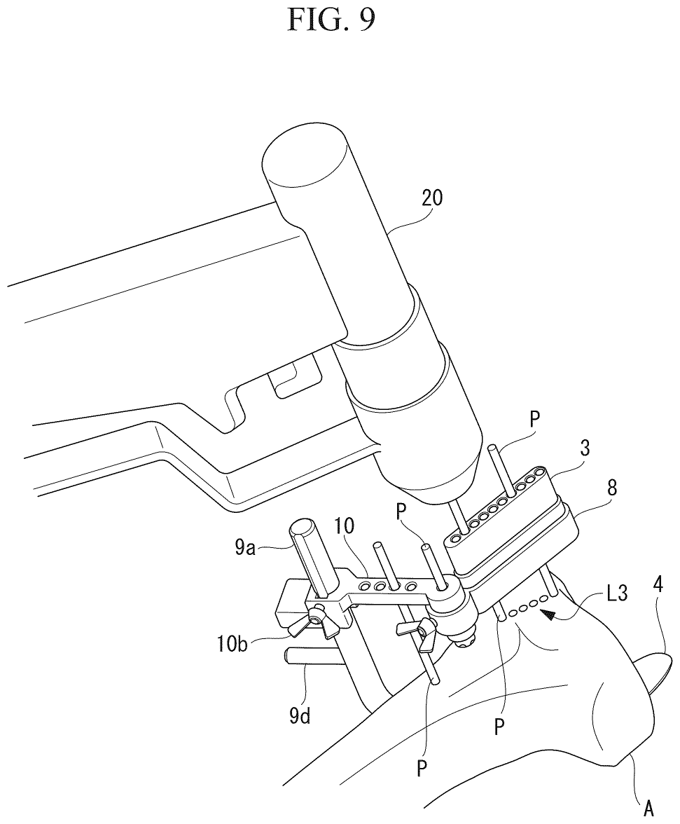

As shown in FIGS. 2A and 2B, the guide portions 2, 3 are substantially flat members having a rectangular shape, and have the same or substantially the same outer shape. The guide portion 2 has a guide hole 2a into which the bone cutting tool T, such as a bone chisel, is inserted, and is used when the tibia A is cut with the bone cutting tool T (see FIG. 10). The guide portion 3 has a plurality of pin holes 3a into which guide pins P are inserted, and is used when a plurality of bone holes forming a perforation L3 are opened in the tibia A by means of the guide pins P before the tibia A is cut (see FIG. 9).

The guide hole 2a is a long hole that penetrates the first guide portion 2 in the first direction and that extends in the second direction, and has a width slightly larger than the thickness of the bone cutting tool T. The second direction is a direction intersecting the first direction, and is preferably a direction orthogonal to the first direction. The first guide portion 2 guides the bone cutting tool T in the guide hole 2a in the first direction and the second direction.

The plurality of pin holes 3a are round holes that individually penetrate the second guide portion 3 in the first direction and that are arranged at intervals in the second direction, and each have an outer diameter slightly larger than the outer diameter of the guide pin P. The second guide portion 3 guides the guide pins P in the pin holes 3a in the first direction.

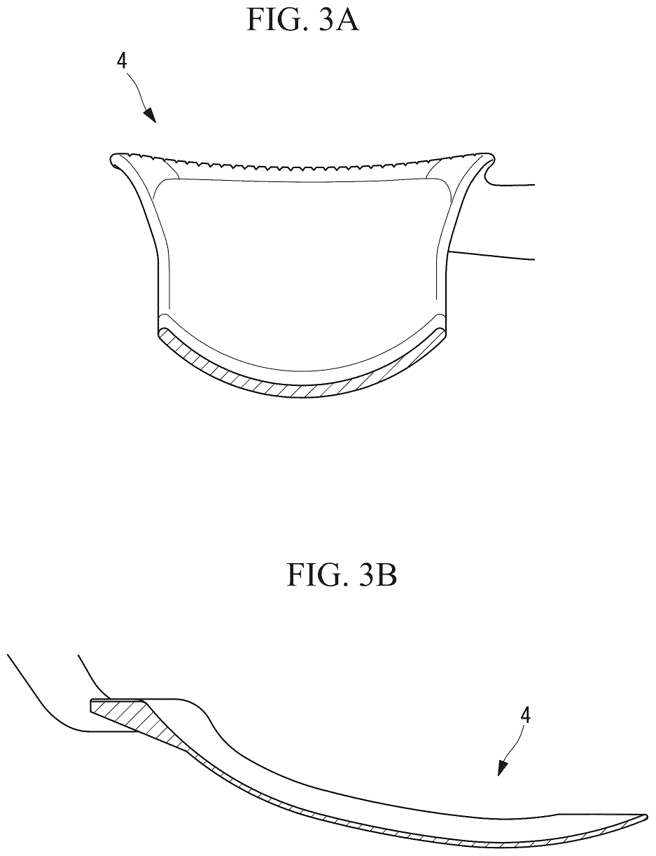

The retractor portion 4 is disposed between the back surface of the tibia A and the soft tissue, and protects the soft tissue from the distal ends of the bone cutting tool T and the guide pins P projecting from the back surface of the tibia A. The retractor portion 4 is formed of a thin strip-shaped member arranged in the second direction, and has a uniform thickness as shown in FIGS. 3A and 3B. FIG. 3A is a cross-sectional view in which the retractor portion 4 is cut in the width direction, and FIG. 3B is a longitudinal sectional view in which the retractor portion 4 is cut along a central axis in the longitudinal direction. In a plan view in the first direction, the retractor portion 4 is disposed in a region overlapping at least the guide hole 2a or the plurality of pin holes 3a, and is preferably disposed in a region larger than the guide portion 2, 3 in order to more reliably protect the soft tissue.

As shown in FIGS. 3A and 3B, the retractor portion 4 has a spoon-like curved shape that is curved in the longitudinal direction and the width direction, and that is convex on the side opposite to a holding portion 8. With such a curved shape, the retractor portion 4 can be disposed along the curved back surface of the tibia A, and it is also possible to prevent the distal ends of the bone cutting tool T and the guide pins P from coming off the retractor portion 4.

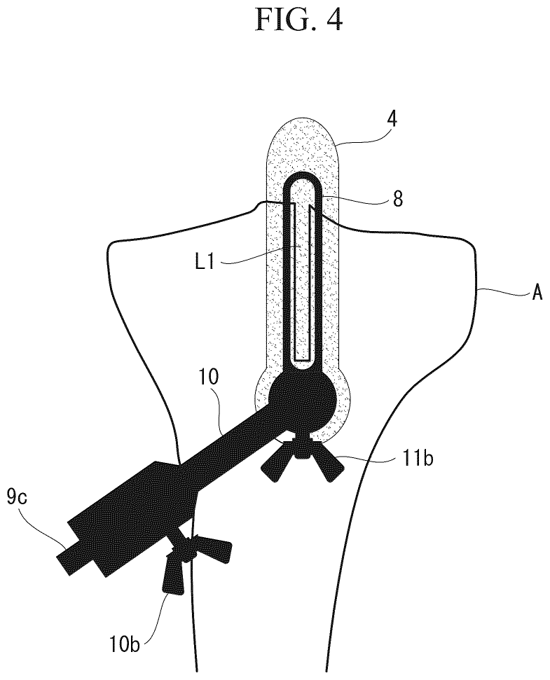

As shown in FIG. 4, in order to enable simultaneous X-ray observation of the retractor portion 4 disposed on the back side of the tibia A and the osteotomy line L1 formed by the bone cutting tool T, the retractor portion 4 preferably has X-ray permeability. For example, in a case in which the retractor portion 4 is formed from stainless steel, the retractor portion 4 preferably has a uniform thickness of 0.5-0.8 mm.

The connection portion 5 has: the holding portion 8 that alternatively holds the two types of guide portions 2, 3; a substantially L-shaped support column portion 9; a beam-shaped support portion 10 that extends from the support column portion 9 in a direction intersecting the first direction; and an attachment portion 11 that is provided at a distal end portion of the support portion 10 and to which the holding portion 8 is attached.

The holding portion 8 is a frame-shaped member extending in the second direction, and has a fitting hole 8a into which the guide portion 2, 3 is fitted in the first direction. Each of the guide portions 2, 3 has a protrusion 2b, 3b provided on the outer surface thereof. As a result of the protrusion 2b, 3b being caught by a surface of the holding portion 8 on the side opposite to the retractor portion 4, the guide portion 2, 3 inserted into the fitting hole 8a is held by the holding portion 8. The guide portion 2, 3 can be easily removed from the holding portion 8 simply by lifting the guide portion 2, 3 to the side opposite to the retractor portion 4. The holding portion 8 may have another structure as long as the holding portion 8 is capable of holding the guide portion 2, 3 in a replaceable manner.

As described above, the first guide portion 2 and the second guide portion 3 are held at the same position by the holding portion 8, and the guide hole 2a and a row of the plurality of pin holes 3a are arranged at the same position. Therefore, the position of the perforation L3 formed by the guide pins P and the position of the osteotomy line L1 formed by the bone cutting tool T can be made to coincide with each other.

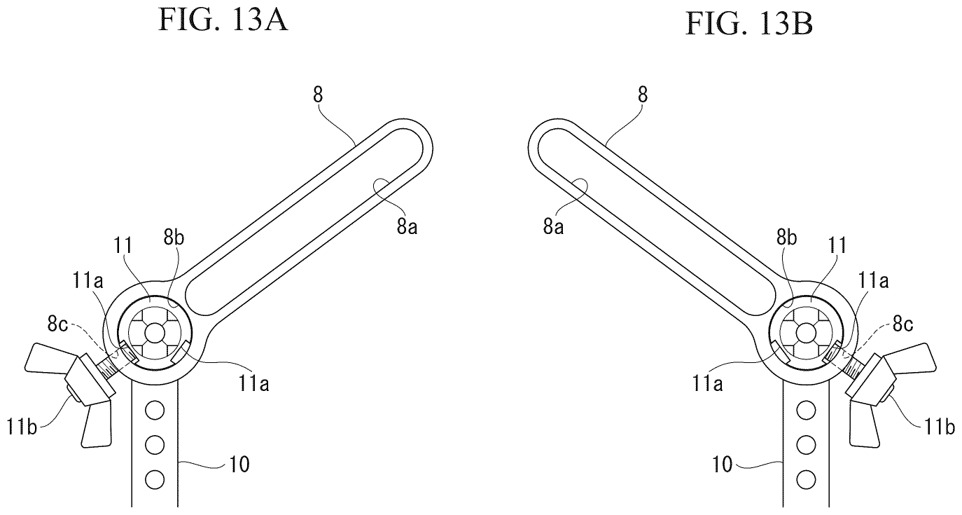

The holding portion 8 further has an attachment hole 8b and a screw hole 8c for attaching the holding portion 8 to the attachment portion 11 (see FIGS. 13A and 13B). The attachment hole 8b is provided at a base end portion of the holding portion 8 and penetrates in the first direction. The screw hole 8c penetrates, in a radial direction, a cylindrical wall surrounding the attachment hole 8b.

The support column portion 9 has: a straight first portion 9a that extends in the first direction; and a second portion 9b that extends from an end portion of the first portion 9a on the retractor portion 4 side in a direction intersecting the first direction. The retractor portion 4 is fixed to the distal end of the second portion 9b.

The support portion 10 extends in the same direction as the second portion 9b, and a hole 10a penetrating in the first direction is provided at a base end portion of the support portion 10. As a result of the first portion 9a extending through the hole 10a, the support portion 10 is supported by the support column portion 9 so as to be movable in the first direction.

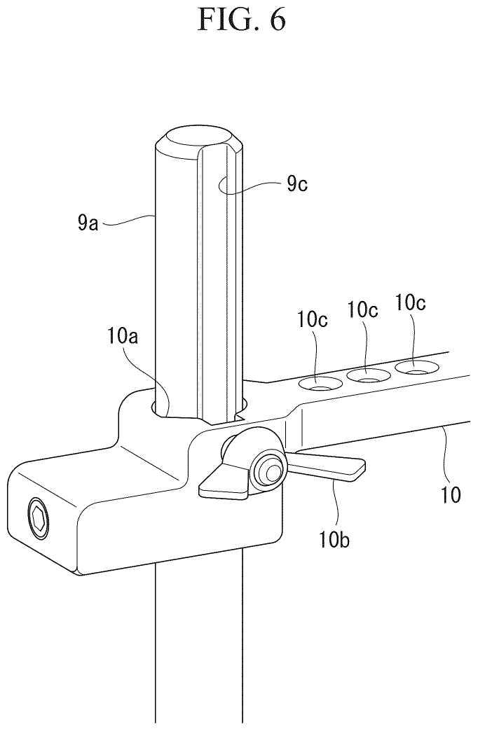

The base end portion of the support portion 10 is provided with a screw hole into which a fixing screw 10b is inserted toward a central axis of the hole 10a, and a groove 9c (see FIG. 6) extending in the first direction and receiving the tip of the fixing screw 10b is formed on the outer circumferential surface of the first portion 9a. The groove 9c is for setting, with respect to the first portion 9a, the angle of the support portion 10 around a longitudinal axis of the first portion 9a to a prescribed angle. The support portion 10 is adjusted, with respect to the first portion 9a, to an angle at which the tip of the fixing screw 10b is fitted into the groove 9c, and the fixing screw 10b is fastened, whereby the support portion 10 is fixed to the first portion 9a at a prescribed angle so that the support portion 10 extends in the same direction as the second portion 9b.

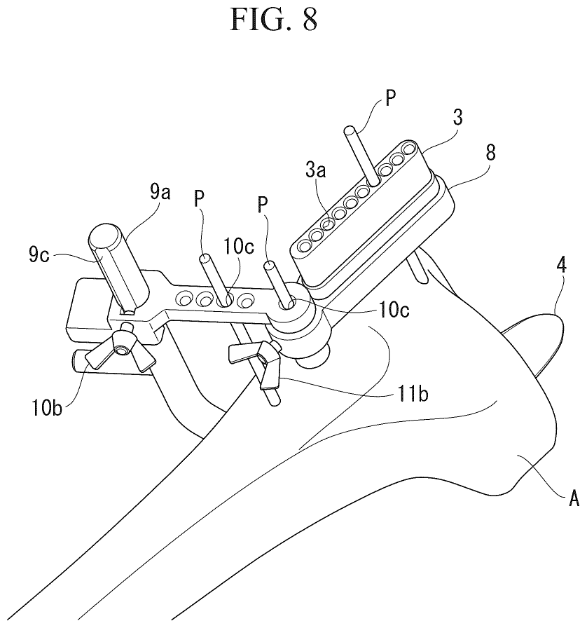

The support portion 10 may have a plurality of pin holes 10c that are arranged in the longitudinal direction of the support portion 10 and that individually penetrate the support portion 10 in the first direction. As a result of the guide pins P being inserted into the tibia A through the pin holes 10c, the guide device 1 can be fixed to the tibia A (for example, see FIG. 8).

The attachment portion 11 is a columnar or tubular member arranged in the first direction, and is fixed to the distal end portion of the support portion 10. The holding portion 8 is fixed to the attachment portion 11 by inserting the attachment portion 11 into the attachment hole 8b and fastening an attachment screw 11b passing through the screw hole 8c.

Grooves 11a each extending in the first direction and receiving the tip of the attachment screw 11b are formed on the outer circumferential surface of the attachment portion 11 (see FIGS. 13A and 13B). The grooves 11a are for setting the angle of the holding portion 8 with respect to the support portion 10. The holding portion 8 is adjusted, with respect to the attachment portion 11, to an angle at which the tip of the attachment screw 11b is fitted into the groove 11a, and the attachment screw 11b is fastened, whereby the holding portion 8 is fixed with respect to the support portion 10 at a prescribed angle so that the holding portion 8 is disposed at a position so as to face the retractor portion 4 in the first direction.

The fixing portion 6 has a first clamping portion 6a and a second clamping portion 6b that face each other in the first direction and that sandwich the tibia A in the front-back direction.

The first clamping portion 6a disposed on the front side of the tibia A indicates one or more projections protruding toward the retractor portion 4 side. The one or more projections 6a are provided at the end of the attachment portion 11 protruding from the attachment hole 8b to the retractor portion 4 side, and are fixed with respect to the support portion 10.

The second clamping portion 6b disposed on the back side of the tibia A indicates a contact surface that comes into contact with the back surface of the tibia A, and is disposed closer to the guide portion 2, 3 than the retractor portion 4 is in the first direction. The contact surface 6b may be a flat surface or a curved surface. The second clamping portion 6b is formed integrally with the retractor portion 4.

By clamping the tibia A with the clamping portions 6a, 6b, the retractor portion 4, the attachment portion 11, the holding portion 8, and the guide portion 2, 3 are fixed with respect to the tibia A. Furthermore, as a result of the projections 6a biting into the surface of the tibia A, the attachment portion 11, the holding portion 8, and the guide portion 2, 3 are more firmly fixed with respect to the tibia A.

In order to further stabilize the position of the fixing portion 6 with respect to the tibia A, the pin hole 10c at the most distal end of the support portion 10 may penetrate the support portion 10 and the attachment portion 11. By inserting the guide pin P into the tibia A through the attachment portion 11, it is possible to more reliably prevent the positional displacement of the holding portion 8 and the guide portion 2, 3 with respect to the tibia A (for example, see FIG. 8).

The adjustment portion 7 is constituted of the support portion 10 that is movable in the first direction, and a lock mechanism that fixes and releases the support portion 10 to and from the support column portion 9. By moving the first clamping portion 6a together with the support portion 10 in the first direction, and by fixing the support portion 10 to the support column portion 9 by means of the lock mechanism, it is possible to adjust the distance between the clamping portions 6a, 6b so that the clamping portions 6a, 6b firmly clamp the tibia A. In order to facilitate the operation of the support portion 10, a handle 9d may be provided on the first portion 9a.

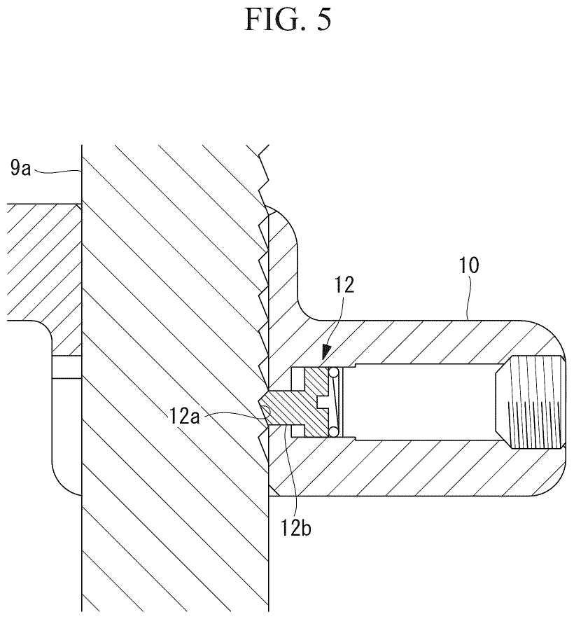

As shown in FIG. 5, an example of the lock mechanism is a ratchet mechanism 12 provided in the support column portion 9 and the support portion 10. The ratchet mechanism 12 has: a plurality of grooves 12a that are formed on the outer surface of the first portion 9a and that are arranged in the first direction; and a projection 12b that is held by the support portion 10 and that engages with the groove 12a, and the projection 12b is urged toward the first portion 9a. The ratchet mechanism 12 restricts the movement of the support portion 10 with respect to the support column portion 9 only in a direction approaching the retractor portion 4, and also holds the support portion 10 at prescribed intervals.

As shown in FIG. 6, another example of the lock mechanism is a stopper that mechanically fixes the support portion 10 to the support column portion 9. The stopper is the fixing screw 10b described above, and by turning the fixing screw 10b, the support portion 10 is fixed to or released from the support column portion 9. The stopper may be in a form other than the fixing screw 10b.

In order to more firmly fix the support portion 10 to the support column portion 9, the lock mechanism may have both the ratchet mechanism 12 and the stopper 10b.

Next, a method of using the guide device 1 will be described with reference to FIGS. 7 to 11.

FIGS. 7 to 10 each show a perspective view in which the tibia A is viewed obliquely from the front side, and FIG. 11 is a side view in which the tibia A is viewed from the outer side.

As shown in FIG. 7, the retractor portion 4 is inserted between the back surface of the tibia A and the soft tissue. Subsequently, the distance between the clamping portions 6a, 6b is adjusted by the adjustment portion 7 so that the clamping portions 6a, 6b firmly clamp the tibia A. By doing so, the holding portion 8 and the retractor portion 4 are fixed with respect to the tibia A by means of the fixing portion 6.

If necessary, as shown in FIG. 8, two guide pins P are inserted into the tibia A through the pin holes 10c. By doing so, the entire guide device 1 is more firmly fixed to the tibia A. As described above, one guide pin P is preferably inserted into the pin hole 10c at the most distal end.

Next, as shown in FIG. 8, the second guide portion 3 is fitted into the fitting hole 8a to be held by the holding portion 8.

Next, as shown in FIG. 9, a drill 20 is used to open a bone hole in the tibia A by means of the guide pin P extending through the pin hole 3a of the second guide portion 3. By changing the pin hole 3a and opening a plurality of bone holes, a perforation L3 consisting of the plurality of bone holes arranged in the second direction is formed. At this time, the pin hole 3a restricts the movement of the guide pin P only in the first direction. Therefore, each bone hole can be formed along the first direction.

Next, as shown in FIGS. 10 and 11, the second guide portion 3 is replaced with the first guide portion 2, the bone cutting tool T, such as a bone chisel, is inserted into the guide hole 2a of the first guide portion 2, and a vertical osteotomy is performed by cutting the tibia A along the perforation L3. At this time, the guide hole 2a restricts the movement of the bone cutting tool T only in the first direction and the second direction. Therefore, it is possible to accurately move the bone cutting tool T along the perforation L3.

As described above, with this embodiment, the bone cutting tool T for performing the vertical osteotomy is guided by the first guide portion 2 in the first direction and the second direction. With this configuration, it is possible to accurately move the bone cutting tool T without displacement, and to accurately perform the osteotomy in the tibia A. In addition, because the retractor portion 4 is present between the back surface of the tibia A and the soft tissue, the soft tissue is protected from the distal end of the bone cutting tool T and the distal ends of the guide pins P projecting from the back surface of the tibia A.

As described above, it is possible to simultaneously achieve an accurate vertical osteotomy and protection of the soft tissue on the back side of the tibia A in TCVO.

In addition, the first guide portion 2 and the second guide portion 3 are held by the holding portion 8 in a replaceable manner, and a row of the pin holes 3a is arranged at the same position as the guide hole 2a. Therefore, the bone cutting tool T inserted into the guide hole 2a is naturally aligned with the perforation L3. Thus, it is possible to easily perform the osteotomy in the tibia A along the perforation L3.

As described above, the guide device 1 for a left leg and a guide device for a right leg have structures symmetrical to each other with respect to a plane in the first direction. A portion of the guide device 1 may be used for both the left leg and the right leg.

Specifically, as shown in FIGS. 12A and 12B, the guide device 1 may be provided with a first unit 1A for both the left leg and the right leg, a second unit 1L for the left leg, and a second unit 1R for the right leg.

The first unit 1A includes the guide portion 2, 3, the first clamping portion 6a, the holding portion 8, the support portion 10, the attachment portion 11, and the screws 10b, 11b. The second units 1L, 1R each include the retractor portion 4, the second clamping portion 6b, and the support column portion 9. The two second units 1L, 1R are symmetrical as a whole with respect to a plane (symmetry plane) in the first direction. The first unit 1A can be attached to and detached from each of the second units 1L, 1R by inserting and removing the first portion 9a into and from the hole 10a of the support portion 10. In addition, the first unit 1A can be attached to either of the two second units 1L, 1R such that the first unit 1A attached to the second unit 1L for the left leg and the first unit 1A attached to the second unit 1R for the right leg are symmetrical to each other with respect to the symmetry plane.

Specifically, the support portion 10 can be attached to the first portion 9a for the left leg and the first portion 9a for the right leg in different orientations by rotating the support portion 10 around the axis in the first direction. In FIGS. 12A and 12B, in order to enable the support portion 10 to be fixed to the first portion 9a for the right leg by using the same fixing screw 10b, the groove 9c of the first portion 9a for the right leg is provided at a position shifted, with respect to the groove 9c of the first portion 9a for the left leg, by 180° about the longitudinal axis of the first portion 9a.

As shown in FIGS. 13A and 13B, the angle of the holding portion 8 with respect to the support portion 10 can be changed between two angles at which the holding portion 8 is symmetrically arranged with respect to a longitudinal axis of the support portion 10 in a plan view as viewed in the first direction. FIGS. 13A and 13B show the holding portion 8 and the attachment portion 11 as viewed from the retractor portion 4 side in the first direction. In a configuration example, the attachment portion 11 has the grooves 11a at two locations in the circumferential direction. With this configuration, it is possible to attach the holding portion 8 to the attachment portion 11 at two angles so that the holding portion 8 faces the retractor portion 4 of either second unit 1L, 1R in the first direction.

It is not preferable that structures such as screws are provided in the retractor portion 4 and the second portion 9b that are inserted into the body. By providing the second units 1L, 1R dedicated for the left leg and the right leg, respectively, it is possible to simplify the structure of the second unit 1L, 1R including the retractor portion 4 and the support column portion 9. For example, as a result of the second portion 9b being welded to a member constituting the retractor portion 4 and the second clamping portion 6b, each of the second units 1L, 1R can be formed of a single member.

Meanwhile, by employing the first unit 1A disposed outside the body for both the left leg and the right leg, it is possible to use the guide device 1 for both the left leg and the right leg.

In the abovementioned embodiment, in order to make the guide device 1 usable for both the left leg and the right leg, the holding portion 8, the support portion 10, and the attachment portion 11 are composed of separate components and are assembled by means of the screws 10b, 11b; however, in a case in which the guide device 1 is dedicated for the left leg or the right leg, the holding portion 8, the support portion 10, and the attachment portion 11 may be integrally formed, for example, by being connected by means of welding or the like.

Although the second clamping portion 6b is integrated with the retractor portion 4 in the abovementioned embodiment, alternatively, the second clamping portion 6b may be separated from the retractor portion 4. In this case, the second clamping portion 6b may be movable with respect to the retractor portion 4 and the support column portion 9 in the first direction, and the distance between the clamping portions 6a, 6b may be adjusted by moving at least one of the clamping portions 6a, 6b in the first direction.

Although the guide device 1 includes the two types of guide portions 2, 3 in the abovementioned embodiment, alternatively, only the guide portion 2 may be provided. In this case, the guide portion 2 may be formed integrally with the connection portion 5, for example, by being directly fixed to the support portion 10 by means of welding.

The present disclosure affords an advantage in that it is possible to simultaneously achieve an accurate vertical osteotomy and protection of soft tissue on the back side of the tibia in TCVO.

### REFERENCE SIGNS LIST

1 guide device
1A first unit
1L, 1R second unit
2, 3 guide portion
2a guide hole
3a pin hole
4 retractor portion
5 connection portion
6 fixing portion
6a first clamping portion
6b second clamping portion
7 adjustment portion
8 holding portion (connection portion)
9 support column portion (connection portion)
10 support portion (connection portion)
A tibia
T bone cutting tool
P guide pin

1. A guide device comprising:
a guide portion disposed on a front side of the tibia, the guide portion guiding a bone cutting tool for cutting the tibia;
a retractor portion disposed on a back side of the tibia, the retractor portion being arranged so as to be spaced apart from the guide portion in a first direction;
a connection portion that connects the guide portion and the retractor portion; and
a fixing portion that fixes the guide portion with respect to the tibia,
wherein the guide portion has a guide hole into which the bone cutting tool is inserted, the guide hole penetrating the guide portion in the first direction and extending in a second direction intersecting the first direction, and
the retractor portion is disposed in a region overlapping at least the guide hole in a plan view in the first direction.

2. The guide device according to claim 1, further comprising another guide portion that guides a pin, wherein
the other guide portion has a plurality of pin holes into which the pin is inserted, the plurality of pin holes individually penetrating the other guide portion in the first direction and being arranged in the second direction, and
the connection portion has a holding portion that alternatively holds the guide portion and the other guide portion such that the guide hole and the plurality of pin holes are arranged at a same position.

3. The guide device according to claim 1, wherein
the fixing portion has a first clamping portion and a second clamping portion that face each other in the first direction and that sandwich the tibia in a front-back direction, and
an adjustment portion that adjusts a distance between the first clamping portion and the second clamping portion in the first direction is further provided.

4. The guide device according to claim 3, wherein
the connection portion has a support column portion that extends in the first direction, and a support portion that is supported by the support column portion so as to be movable in the first direction,
the first clamping portion is fixed with respect to the support portion, and
the adjustment portion has a lock mechanism that fixes and releases the support portion to and from the support column portion.

5. The guide device according to claim 1, further comprising a first unit, a second unit for a left leg, and a second unit for a right leg, wherein
the first unit comprises the guide portion and a portion of the connection portion,
the second unit for the left leg and the second unit for the right leg each comprises the retractor portion, and another portion of the connection portion to and from which the aforementioned portion of the connection portion is attachable and detachable,
the second unit for the left leg and the second unit for the right leg are symmetrical to each other with respect to a plane in the first direction; and
the first unit can be attached to either of the second unit for the left leg and the second unit for the right leg.
